(19) United States
(12) Patent Application Publication
(10) Pub. No.: US 2022/0362323 A1
(43) Pub. Date: Nov. 17, 2022

(54) SYSTEMS AND METHODS FOR PRODUCING A KAVA LIQUID DIETARY SUPPLEMENT

(71) Applicant: Kula LLC, Santa Monica, CA (US)

(72) Inventor: Sol Cho Broady, Santa Monica, CA (US)

(21) Appl. No.: 17/358,553

(22) Filed: Jun. 25, 2021

Related U.S. Application Data
(63) Continuation-in-part of application No. 17/246,698, filed on May 2, 2021.

(51) Int. Cl. A61K 36/67; A61K 9/00; A61K 47/46; A61K 47/12; A61K 45/06; A23L 33/105; A23L 33/125; A23L 33/15

(52) U.S. Cl. CPC A61K 36/67; A61K 9/0095; A61K 47/46; A61K 47/12; A61K 45/06; A23L 33/105; A23L 33/125; A23L 33/15; A61K 2236/53; A23V 2002/00

(57) ABSTRACT

A method for preparing a liquid dietary supplement includes extracting kavalactones from a Kava plant, heating water to approximately 160° Fahrenheit, and mixing kavalactones with sugar in a 1:2 ration creating a Kava/sugar mixture. The method also includes pouring Kava/sugar and heated water into a mixer, mixing, for approximately twenty (20) minutes, and pumping mixed Kava/sugar through a filter bag generating a slurry. Further the method includes adding the slurry to water heated to 55° Fahrenheit and agitating the slurry and the water heated to 55° with a mixer.

### CROSS-REFERENCE TO RELATED APPLICATIONS

This application is a Continuation-In-Part Application of U.S. application Ser. No. 17/246,698, filed on May 2, 2021, entitled SYSTEMS AND METHODS FOR PRODUCING A KAVA LIQUID DIETARY SUPPLEMENT, which is herein incorporated by reference in its entirety.

### BACKGROUND

Kava or Kava Kava (Piper methysticum) is a tropical plant grown in the Pacific Islands, and Kava is often exported as an herbal medicine. Kava is known for its many health benefits. As an example, Kava has a sedating effect when ingested. Also, the root of the plant is used to produce a drink with sedative, anesthetic, and euphoriant properties. It is often noted as beneficial for treating anxiety.

The active ingredients in Kava are called kavalactones. The kavalactones are a class of lactone compounds found in the kava shrub. Kavalactones are by their nature sticky and insoluble.

These properties prove difficult in manufacturing of products containing kavalactones. In this regard, when a solution containing kavalactones is introduced to equipment, e.g., filter machinery, the sticky kavalactones interfere with the processing. Thus, it is difficult to create products containing kavalactones.

### SUMMARY

Various disclosed embodiments include cover assemblies for an electrical busbar connection, busbar connector assemblies, and battery systems.

In some embodiments a method for preparing a liquid dietary supplement include extracting kavalactones from a Kava plant, heating water to approximately 160° Fahrenheit, and mixing kavalactones with sugar in a 1:2 ration creating a Kava/sugar mixture. The method also includes pouring Kava/sugar and heated water into a mixer, mixing, for approximately twenty (20) minutes, and pumping mixed Kava/sugar through a filter bag generating a slurry. Further the method includes adding the slurry to water heated to 55° Fahrenheit and agitating the slurry and the water heated to 55° with a mixer.

Illustrative embodiments of a liquid dietary supplement include water, organic cane sugar, a stabilizer, and Kava Kava root extract. The liquid dietary supplement also includes a vitamin mineral blend, citric acid, and stevia extract.

Other illustrative embodiments of a system for preparing a liquid dietary supplement include a means for extracting kavalactones from a Kava plant, a means for heating water to approximately 160° Fahrenheit, and a means for mixing kavalactones with sugar in a 1:2 ration creating a Kava/sugar mixture. The method also includes a means for pouring Kava/sugar and heated water into a mixer, a means for mixing, for approximately twenty (20) minutes, and a means for pumping mixed Kava/sugar through a filter bag generating a slurry. Further the method includes a means for adding the slurry to water heated to 55° Fahrenheit and a means for agitating the slurry and the water heated to 55° with a mixer.

The foregoing summary is illustrative only and is not intended to be in any way limiting. In addition to the illustrative aspects, embodiments, and features described above, further aspects, embodiments, and features will become apparent by reference to the drawings and the following detailed description.

### BRIEF DESCRIPTION OF THE DRAWINGS

The present disclosure can be better understood with reference to the following drawing figures. The elements of the drawing figures are not necessarily to scale relative to each other, emphasis instead being placed upon clearly illustrating the principles of the disclosure. Furthermore, like reference numerals designate corresponding parts throughout the several views.

FIG. 1 is a flowchart depicting the method of producing a beverage containing Kava in accordance with an embodiment of the present disclosure.

FIG. 2 is an exemplary filter housing used in producing a liquid dietary supplement containing Kava in accordance with an embodiment of the present disclosure

FIG. 3 is an exemplary high shear mixer used in the producing a liquid dietary supplement containing Kava in accordance with an embodiment of the present disclosure.

FIG. 4 is an exemplary cross-sectional view of a sanitary bag filtration device in accordance with an embodiment of the present disclosure.

FIG. 5 is a table depicting a formula for a Kava-containing liquid dietary supplement having a lemon ginger flavor in accordance with an embodiment of the present disclosure.

FIG. 6 is a table depicting a formula for a Kava-containing liquid dietary supplement having a tango berry flavor in accordance with an embodiment of the present disclosure.

FIG. 7 is a table depicting a formula for a Kava-containing liquid dietary supplement having a blackberry orange flavor in accordance with an embodiment of the present disclosure.

FIG. 8 is a table depicting a formula for a Kava-containing liquid dietary supplement having a tango berry flavor in accordance with an embodiment of the present disclosure.

### DETAILED DESCRIPTION

The present disclosure describes systems and methods for producing a liquid dietary supplement containing Kava in accordance with an embodiment of the present disclosure. In accordance with exemplary embodiments, the systems and methods may first include the manufacture of Kava Kava root PE comprising 30% Kava lactones [symbol] LC. This process may include first grinding plant material, e.g., Piper methysticum F. Forst using 95% Ethanol and 5% water, Kava is extracted from the plant material and may be filtered producing a cake substance. Note that the plant extract (cake) produced is a substance with desirable properties.

Next, concentration and desolventation may be performed on the cake material using 95% Ethanol and 5% water. Native extraction occurs so that the material consisting only of Kava Kava is created, excluding any excipients or other added substances. The native extraction process may utilize multiple passes of the 95% Ethanol and 5% water solution through the cake material. Each successive pass extracts more kavalactones, until the desired 30% concentration is reached.

The created substance may then be spray-dried using modified starch and silicon dioxide. The substance created may then be sieved. That is, the sieving process filters large particles out of the substance to ensure that the large particles do not interfere with other machinery, e.g., a pump. The substances created in this process are packaged and labelled thereby creating samples of the Kava-containing substance. Tests may be performed on the substance for quality control, general aspect, analytical quality, and microbiological quality. The substance produced is Kava Kava root PE having 30% kavalactones tested via High-performance liquid chromatography (HPLC). The extraction method described may be varied in many different ways and with differing orders of steps without departing from the scope of the disclosure.

Once the kavalactones are extracted successfully from the plant, the kavalactones may be used to create a liquid dietary supplement. In this regard, the first step is heating water to 160° F. In one embodiment, the water to kavalactones ratio may be 10:1. The next step is mixing the kavalactones with sugar. In one embodiment, the kavalactones to sugar ratio may be 1:2. The mixture of sugar, heated water, and kavalactones is poured into a vessel and the mixture is pulverized with a high shear mixer. The solution may be mixed for twenty (20) minutes. The mixture may then pumped through a fifty (50) micron filter. The output of the fifty (50) micron filter is a liquid dietary supplement consumable by humans. All of the ratios and filter sizes may be varied without departing from the scope of the disclosure.

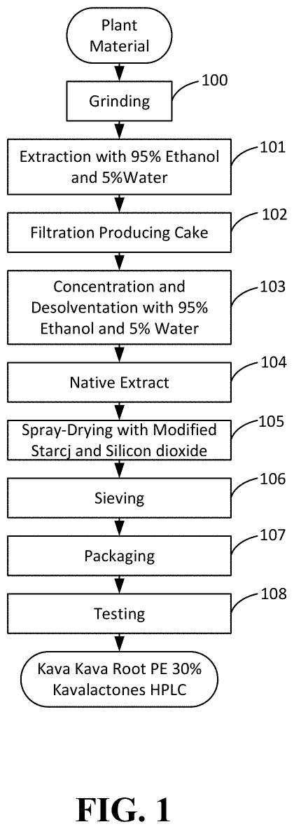

FIG. 1 is an exemplary flowchart depicting the extraction of kavalactones from a plant, e.g., Piper methysticum F. Forst. Note that root extracts from Kava Kava are used for the treatment of anxiety and restlessness. The Kava Kava plants are found in the Pacific Islands.

In step 100 the plant material, noble roots only, is grinded. In one embodiment, noble root plant material is grinded with a mill. It may be grinded using other means in other embodiments of the present disclosure.

In step 101 those parts of the plant roots, e.g., kavalactones, are extracted from the ground plant material. In one embodiment, extraction of the kavalactones is effectuated using ninety-five percent (95%) Ethanol and five percent (5%) water. Other substances at other percentages may be used in other embodiments.

In step 102, the solution of kavalactones, water, and ethanol are filtered. When filtered and rested, the solution produces a cake-like substance.

In step 103, the cake-like substance is concentrated and dissolved in ninety-five percent (95%) Ethanol and five percent (5%) water creating a solution of Ethanol, water, and kavalactones. Other concentrations of water and Ethanol may be used in other embodiments.

In step 104, the kavalactones are extracted from the solution via native extraction in the multi pass process described above. In step 105, the kavalactones are spray-dried using modified starch and silicon dioxide. Other spray-drying materials may be used in accordance with other embodiments.

In step 106, sieving is performed on the spray-dried product to obtain the kavalactones. In step 107, the kavalactones can be packaged and labelled, and samples may be created of the kavalactones. Finally, the final product is tested for quality control, general aspect, analytical quality, and microbiological quality in step 108.

Steps 100-106 may be used to generate Kava Kava root PE 30% kavalactones HPLC. The final product may then be used to create a liquid dietary supplement containing the kavalactones.

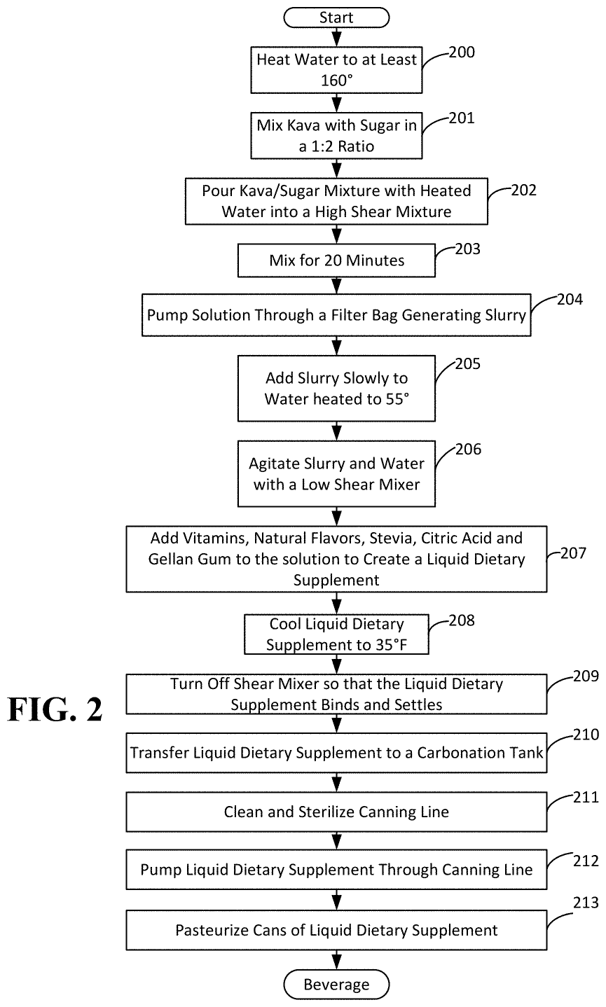

FIG. 2 is a flowchart indicative of the process of creating a liquid dietary supplement containing kavalactones in accordance with an embodiment of the present disclosure. The sequence of steps is not necessarily in the order presented. The liquid dietary supplement may be created in other ways in other embodiments.

In step 200, water is heated to at least 160°. While 160° is one embodiment, other temperatures may be used in other embodiments.

In step 201, kavalactones are mixed with sugar in a 1:2 ratio creating a Kava/sugar mixture. That is, one measurement of kavalactones to two measurements of sugar. Note that while a 1:2 ratio is cited, other ratios may be used in other embodiments.

In step 202, the kava/sugar mixture is mixed with the heated water in a large vessel and pulverized with a high shear blender. Note that a high shear mixer disperses one phase or ingredient (liquid, solid, gas) into a main continuous phase (liquid). The high shear mixer contains at least a rotor or impeller and a stator (or an array of rotors and stators) to mix the kava/sugar mixture in a tank to create shear. The mixture is mixed in the high shear mixer for twenty minutes in step 203.

In step 204 a sanitary bag filter housing is used. In this regard, the kava/sugar mixture is pumped through a filter bag. In one embodiment, a 50 micron filter bag is used. However, other types of bags may be used in other embodiments. The resulting mixture is a non-sticky, evenly dispersed slurry containing kavalactones, which forms the functional base of the liquid dietary supplement. In various embodiments various filtration members or filter bags may be used with a nominal pore size of less than 50 microns. Further, as smaller pore size filters may be used, the liquid slurry may need to be pressurized.

In step 205 the slurry is slowly added to remaining water which has been heated to 55 degrees. The solution is agitated by a low shear mixer in a vessel in step 206. As the slurry becomes soluble in the solution, a functional blend of vitamins, natural flavors, stevia, citric acid, and gellan gum are slowly added into the solution. This forms the liquid dietary supplement. In accordance with various embodiments other additives may be used to create varied desired results in taste, quality, consistency, etc.

In step 208 the liquid dietary supplement is cooled to 35 degrees. The low shear mixer is turned off, and the liquid mixture binds and settles in step 209 after some time.

In step 210 the liquid dietary supplement is transferred from the blending vessel to the carbonation tank, where it remains at 35 degrees for 5-8 hours of carbonation. The liquid dietary supplement is uniformly carbonated to 3.0 volumes.

In step 211 the canning line is cleaned and sterilized to ensure quality. In step 212, the liquid dietary supplement is pumped via an electric pump at 12 psi through the canning line for 8 hours of processing. The liquid dietary supplement is kept at 35 degrees throughout this process.

In step 213 the liquid dietary supplement, now in 12 oz sleek cans, is pasteurized at 160 degrees for 10 minutes. The preferred method of pasteurization is tunnel, though other forms may be used in other embodiments, such as but not limited to batch Pasteurization

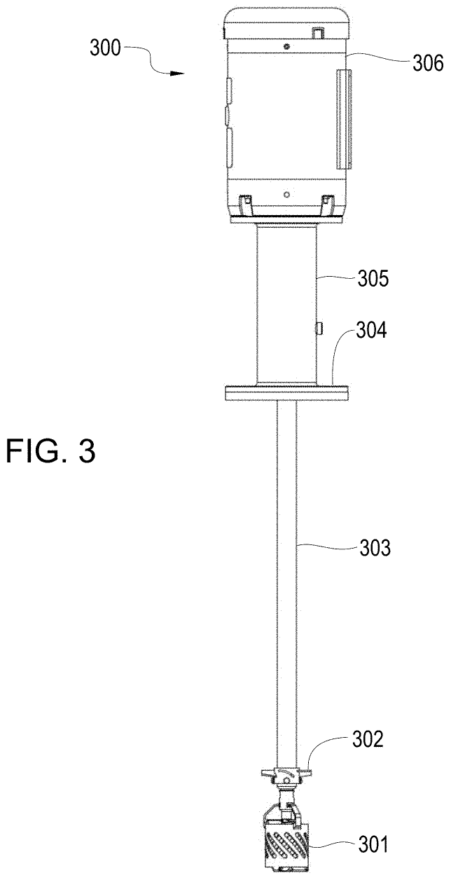

FIG. 3 is a high shear mixer 300 used to generate the liquid dietary supplement in accordance with an embodiment of the present disclosure. The high shear mixer 300 comprises a head 301, an impeller 302, a shaft 303, a seal plate 304, a bearing frame 305, and a motor 306.

In one embodiment, the head 301 is a Rotosolver II. In this regard, the head comprises a first opening (not shown) in the top of the head and a second opening in the bottom of the head. Further, the head 301 comprises machined slots in a diagonal pattern on the outer walls of the head 301. In operation, substance enters a cavity of the head 301 from the top and bottom openings.

As substance enters the top of the head 301, they are exposed to mechanical shears (not shown). The high-velocity, counter current streams converge with a stator within the head 301. This causes high turbulence and hydraulic shear, without momentum loss from obstructions within the stator.

Centrifugal pressure forces the substance to a periphery (not shown) of the stator where it is subjected to further mechanical shear as material passes the sharpened edges of the slots. Notably, the discharge of the substance combines with a slower moving tank flow for additional hydraulic shear.

The impeller 302 is coupled to the head via a shaft. The impeller 302 is a rotor that rotates thereby increasing a pressure and a flow of the substance.

The head 301 and the impeller 302 are coupled to the output shaft 303. The output shaft, when rotated, causes the head 1 to rotate and mix the substance in which the head 301 is contained.

The high shear mixer 300 further comprises a seal plate 304 that is coupled to a bearing frame 305. The bearing frame 305 is rotated by a motor 306. When the motor 306 is on, it rotates the mixer output shaft 303 thereby rotating the head 301 and mixing the substance.

In accordance with various embodiments, various combinations of low shear and high shear blending may be used. Further, the blending may be, in some embodiments, improved with the use of a homogenizer in combination with high shear blending. The use of high shear blending with the addition of the homogenizer and adequate filtration, as discussed above, may lead to a drink having no noticeable sedimentation.

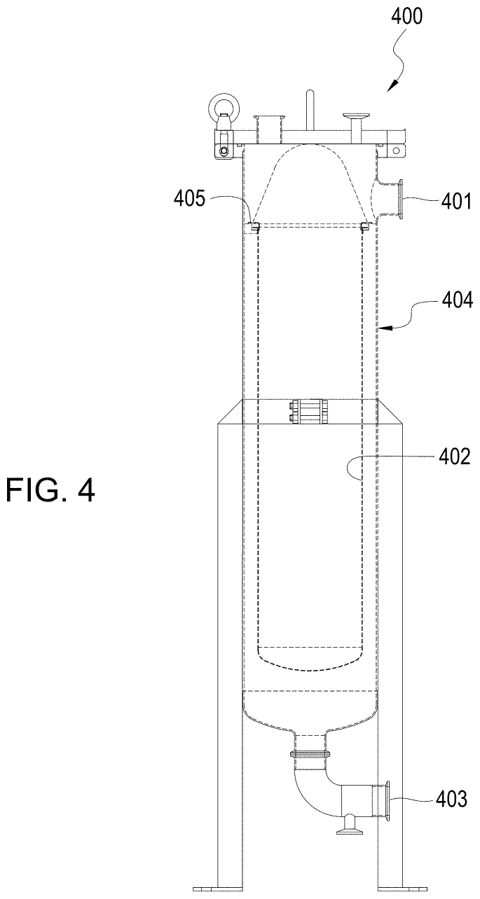

FIG. 4 is across-sectional view of an exemplary sanitary bag filtration device 400 in accordance with an embodiment of the present disclosure. The sanitary bag filtration device 400 comprises a housing 404. The housing 404 comprises an inlet pipe 401, a basket 402, and an outlet pipe 403. The basket sits on and is coupled to a basket seat 405.

In operation, a filter bag (not shown), e.g., a 50 micron filter bag, is placed within the basket 402. Note that the filter bag may rest on and be coupled to the basket seat 405. The kava/sugar mixture described above is introduced to the inlet pile 401. The kava/sugar mixture goes into the basket 402 through the filter bag and exits the housing 402 through an outlet pipe 403. The mixture outputted is a non-sticky, evenly dispersed slurry containing kavalactones, which forms the functional base of the liquid dietary supplement.

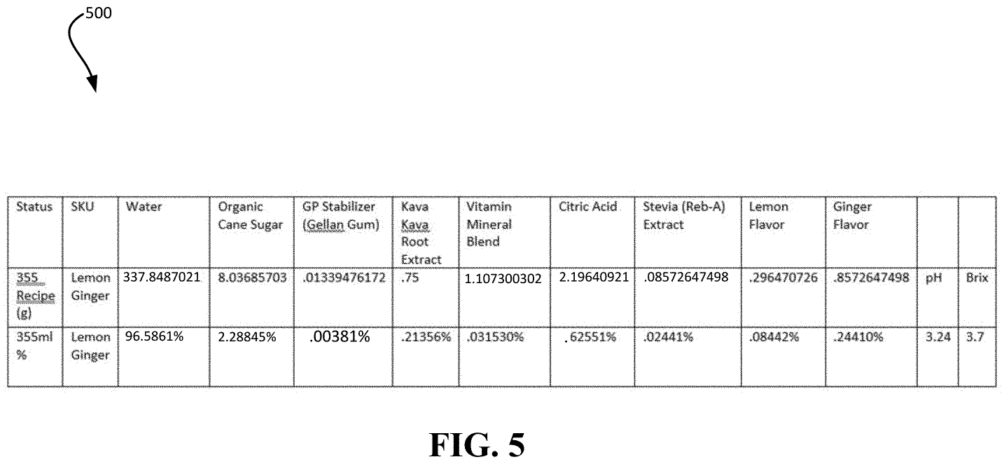

FIG. 5 is a table 500 showing a formula for a Kava-containing liquid dietary supplement having a lemon ginger flavor in accordance with an embodiment of the present disclosure. Notably, the lemon ginger flavored Kava liquid dietary supplement comprises water, organic cane sugar, a stabilizer, e.g., gellan gum, Kava Kava Root extract, a vitamin and mineral blend, citric acid, and stevia. In addition, the lemon ginger flavored Kava liquid dietary supplement has lemon flavoring and ginger flavoring. The lemon ginger flavored Kava liquid dietary supplement has a pH of 3.24 and a Brix of 3.7. Note that Brix identifies the amount of dissolved solids in the liquid via its specific gravity and is used to measure dissolved sugar, especially.

The lemon ginger flavored Kava liquid dietary supplement contains 37.8587021 (approximately 338) grams of water and makes up 95.5861% (approximately 96%) of the lemon ginger flavored Kava liquid dietary supplement. Thus, much of the lemon ginger flavored Kava liquid dietary supplement is water.

The lemon ginger flavored Kava liquid dietary supplement contains 8.03685703 grams (approximately 8 grams) of organic cane sugar and makes up 2.2920% (approximately 2%) of the lemon ginger flavored Kava liquid dietary supplement. Note that the organic cane sugar is sugar obtained directly from sugar cane

The lemon ginger flavored Kava liquid dietary supplement contains 0.01339476182 grams (approximately 0.01 grams) of a stabilizer, e.g., gellan gum, which makes up approximately 0.0038% (0.004%) of the lemon ginger flavored Kava liquid dietary supplement. Note that gellan gum is a water-soluble anionic polysaccharide used to stabilize, thicken, add texture, and suspend other ingredients in the lemon ginger flavored liquid dietary supplement.

The lemon ginger flavored Kava liquid dietary supplement contains 0.75 grams of Kava Kava root extract, which makes up approximately 0.21356% (approximately 0.2%) of the lemon ginger flavored Kava liquid dietary supplement. Kava Kava roots are known for their medicinal properties. For example, the Kava Kava root may be used in treating anxiety disorders, benzodiazepine withdrawal, common cold/upper respiratory tract infections, depression, epilepsy, headaches/migraines, insomnia, musculoskeletal pain, psychosis, and stress.

The lemon ginger flavored Kava liquid dietary supplement contains approximately 1.073999392 grams (approximately 1 gram) of a vitamin and mineral blend, which makes up 0.031530% of the lemon ginger flavored Kava liquid dietary supplement. The vitamins and mineral blend may vary depending upon the application or use of the lemon ginger flavored Kava liquid dietary supplement.

The lemon ginger flavored Kava liquid dietary supplement contains approximately 2.19670921 grams (approximately 2 grams) of citric acid, which makes up approximately 0.062551% (approximately 0.06%) of lemon ginger flavored Kava liquid dietary supplement. Note that the citric acid has a sour taste and is predominantly used as a flavoring and a preserving agent in the lemon ginger flavored Kava liquid dietary supplement.

The lemon ginger flavored Kava liquid dietary supplement contains approximately 0.085726470726 grams (approximately 0.09 grams) of Stevia extract, which makes up approximately 0.02441% (approximately 0.2%) of the lemon ginger flavored Kava liquid dietary supplement. Note that Stevia is natural, unlike other sugar substitutes. It is made from a leaf related to popular garden flowers like asters and chrysanthemums.

To provide the lemon ginger flavored Kava liquid dietary supplement with its lemon ginger flavor, flavors are added to the base recipe. In the example of the lemon ginger flavored Kava liquid dietary supplement, approximately 0.296470726 grams (approximately 0.3 grams), which is about 0.08442% of the lemon ginger flavored Kava liquid dietary supplement, is added to the base formula. In addition, approximately 0.8572647498 grams (approximately 0.9 grams) of ginger flavor is added, which is approximately 0.24410% of the lemon ginger flavored Kava liquid dietary supplement. The pH of the lemon ginger flavored liquid dietary supplement is approximately 3.24 and the Brix is approximately 3.7.

All of the above proportions and quantities of ingredients should not be considered limiting as to the scope of the disclosed embodiments as they are provided by way of example only.

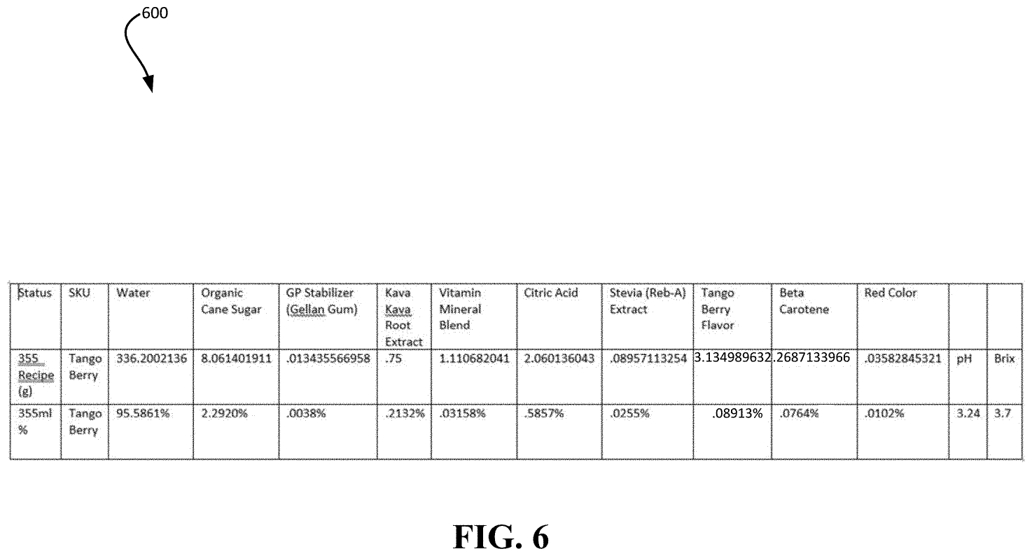

FIG. 6 is a table 600 showing a formula for a Kava-containing liquid dietary supplement having a tango berry flavor in accordance with an embodiment of the present disclosure. Notably, the tango berry flavored Kava dietary supplement comprises water, organic cane sugar, a stabilizer, e.g., gellan gum, Kava Kava Root extract, a vitamin and mineral blend, citric acid, and stevia. In addition, the tango berry flavored Kava liquid dietary supplement container tango berry flavor, beta carotene, and red coloring. The tango berry flavored Kava liquid dietary supplement has a pH of 3.24 and a Brix of 3.7. As noted above Brix identifies the number of dissolved solids in the liquid via its specific gravity and is used to measure dissolved sugar, especially.

The tango berry flavored Kava liquid dietary supplement contains approximately 336.2002136 (approximately 336) grams of water, which makes up 95.5861% (approximately 96%) of the tango berry flavored Kava liquid dietary supplement. Thus, much of the tango berry flavored Kava liquid dietary supplement is water.

The tango berry flavored Kava liquid dietary supplement contains approximately 8.061401911 grams (approximately 8 grams) of organic cane sugar and makes up approximately 2.2920% (approximately 2%) of the tango berry flavored Kava liquid dietary supplement. As noted above, the organic cane sugar is sugar obtained directly from sugar cane

The tango berry flavored Kava liquid dietary supplement contains approximately 0.013435566958 grams (approximately 0.01 grams) of a stabilizer, e.g., gellan gum, which makes up approximately 0.0038% (0.004%) of the tango berry flavored Kava liquid dietary supplement. As noted above, gellan gum is a water-soluble anionic polysaccharide used to stabilize, thicken, add texture, and suspend other ingredients in the lemon ginger flavored liquid dietary supplement.

The tango berry flavored Kava liquid dietary supplement contains 0.75 grams of Kava Kava root extract, which makes up approximately 0.21356% (approximately 0.2%) of the tango berry flavored Kava liquid dietary supplement. As indicated herein, Kava Kava roots are known for their medicinal properties. For example, the Kava Kava root may be used in treating anxiety disorders, benzodiazepine withdrawal, common cold/upper respiratory tract infections, depression, epilepsy, headaches/migraines, insomnia, musculoskeletal pain, psychosis, and stress.

The tango berry flavored Kava liquid dietary supplement contains approximately 1.11068 grams (approximately 1 gram) of a vitamin and mineral blend, which makes up 0.031580% of the tango berry flavored Kava liquid dietary supplement. The vitamins and mineral blend may vary depending upon the application or use of the tango berry flavored Kava liquid dietary supplement.

The tango berry flavored Kava liquid dietary supplement contains approximately 2.060136043 grams (approximately 2 grams) of citric acid, which makes up approximately 0.5857% (approximately 0.6%) of the tango berry flavored Kava liquid dietary supplement. Note that the citric acid has a sour taste and is predominantly used as a flavoring and a preserving agent in the tango berry flavored Kava liquid dietary supplement.

The tango berry flavored Kava liquid dietary supplement contains approximately 0.08957113254 grams (approximately 0.09 grams) of Stevia extract, which makes up approximately 0.0255% (approximately 0.3%) of the tango berry flavored Kava liquid dietary supplement. As noted above, that Stevia is natural, unlike other sugar substitutes. It is made from a leaf related to popular garden flowers like asters and chrysanthemums.

To provide the tango berry flavored Kava liquid dietary supplement with its tango berry flavor, additional substances are added to the base formula. In one example of the tango berry flavored Kava liquid dietary supplement, approximately 3.13498632 grams (approximately 3 grams), which is about 0.08913% (approximately 1%) of tango berry flavored Kava liquid dietary supplement, is added to the base formula. In addition, approximately 0.2687133966 grams (approximately 0.3 grams) of Beta Carotene is added. Note that Beta Carotene is a red-orange pigment found in plants and fruits. Beta Carotene is approximately 0.0764% of the tango berry flavored Kava liquid dietary supplement. Further, red color is added to the tango berry flavored Kava liquid dietary supplement. In this regard, approximately 0.035828445321 grams (approximately 0.04 grams) is added. The pH of the tango berry flavored Kava liquid dietary supplement is approximately 3.24 and the Brix is approximately 3.7.

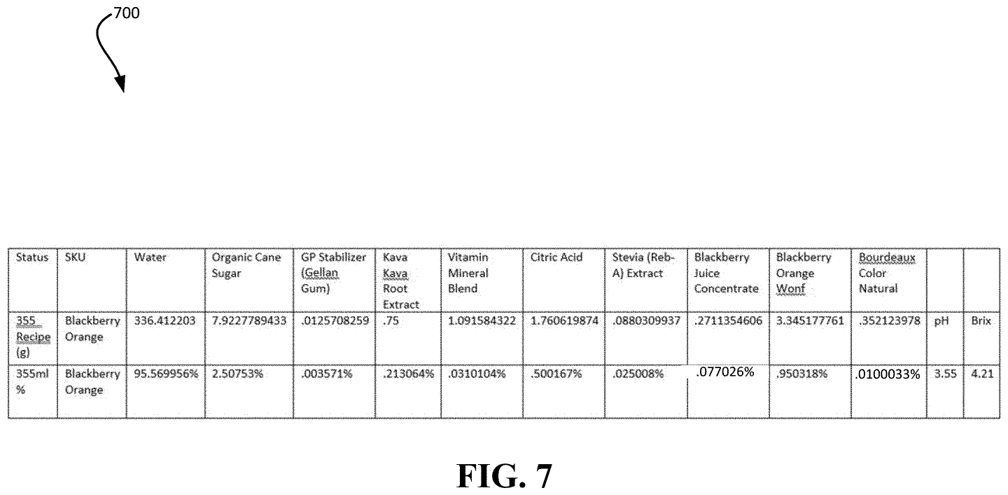

FIG. 7 is a table 700 showing a formula for a Kava-containing liquid dietary supplement having a blackberry orange flavor in accordance with an embodiment of the present disclosure. Notably, the blackberry orange flavored Kava dietary supplement comprises water, organic cane sugar, a stabilizer, e.g., gellan gum, Kava Kava Root extract, a vitamin and mineral blend, citric acid, and stevia. In addition, the blackberry orange flavored Kava dietary supplement contains blackberry juice concentrate, blackberry orange with other natural flavors (WONF), and Bordeaux coloring. The blackberry orange flavored Kava dietary supplement has a pH of 3.55 and a Brix of 4.21. As noted above, Brix identifies the number of dissolved solids in the liquid via its specific gravity and is used to measure dissolved sugar, especially.

The blackberry orange flavored Kava dietary supplement contains approximately 336.412203 (approximately 336) grams of water, which makes up 95.569956% (approximately 96%) of the blackberry orange flavored Kava dietary supplement. Thus, much of the blackberry orange flavored Kava dietary supplement is water.

The blackberry orange flavored Kava dietary supplement contains approximately 7.92277894433 grams (approximately 8 grams) of organic cane sugar and makes up approximately 2.50753% (approximately 2%) of the blackberry orange flavored Kava dietary supplement. As noted above, the organic cane sugar is sugar obtained directly from sugar cane

The blackberry orange flavored Kava dietary supplement contains approximately 0.0125708259 grams (approximately 0.01 grams) of a stabilizer, e.g., gellan gum, which makes up approximately 0.00357% (approximately 0.004%) of the blackberry orange flavored Kava dietary supplement. As noted above, gellan gum is a water-soluble anionic polysaccharide used to stabilize, thicken, add texture, and suspend other ingredients in the blackberry orange flavored Kava dietary supplement.

The blackberry orange flavored Kava dietary supplement contains 0.75 grams of Kava Kava root extract, which makes up approximately 0.213064% (approximately 0.2%) of the. As indicated herein, Kava Kava roots are known for their medicinal properties. For example, the Kava Kava root may be used in treating anxiety disorders, benzodiazepine withdrawal, common cold/upper respiratory tract infections, depression, epilepsy, headaches/migraines, insomnia, musculoskeletal pain, psychosis, and stress.

The blackberry orange flavored Kava dietary supplement contains approximately 1.091584322 grams (approximately 1 gram) of a vitamin and mineral blend, which makes up 0.0310104% (approximately 0.03%) of the blackberry orange flavored Kava dietary supplement. The vitamins and mineral blend may vary depending upon the application or use of the blackberry orange flavored Kava dietary supplement.

The blackberry orange flavored Kava dietary supplement contains approximately 1.760619874 grams (approximately 2 grams) of citric acid, which makes up approximately 0.500167% (approximately 0.6%) of the. As noted above, the citric acid has a sour taste and is predominantly used as a flavoring and a preserving agent in the blackberry orange flavored Kava dietary supplement.

The blackberry orange flavored Kava dietary supplement contains approximately 0.0880309937 grams (approximately 0.09 grams) of Stevia extract, which makes up approximately 0.025008% (approximately 0.3%) of the blackberry orange flavored Kava liquid dietary supplement. As noted above, that Stevia is natural, unlike other sugar substitutes. It is made from a leaf related to popular garden flowers like asters and chrysanthemums.

To provide the blackberry orange flavored Kava liquid dietary supplement with its blackberry orange flavor, additional substances are added to the base formula. In one example of the blackberry orange flavored Kava liquid dietary supplement, approximately 0.2711354606 grams (approximately 0.3 grams), which is approximately 0.0770206% (approximately 0.1%) of blackberry juice is added to blackberry orange flavored Kava liquid dietary supplement. In addition, approximately 3.345177761 grams (approximately 3 grams) of blackberry orange with other natural flavors is added. Further, Bordeaux color is added to the blackberry orange flavored Kava liquid dietary supplement. In this regard, approximately 0.352123978 grams (approximately 0.04 grams) is added, which is about 0.0102 (approximately 0.01) to the blackberry orange flavored Kava liquid dietary supplement. The pH of the blackberry orange flavored Kava liquid dietary supplement is approximately 3.55 and the Brix is approximately 4.21.

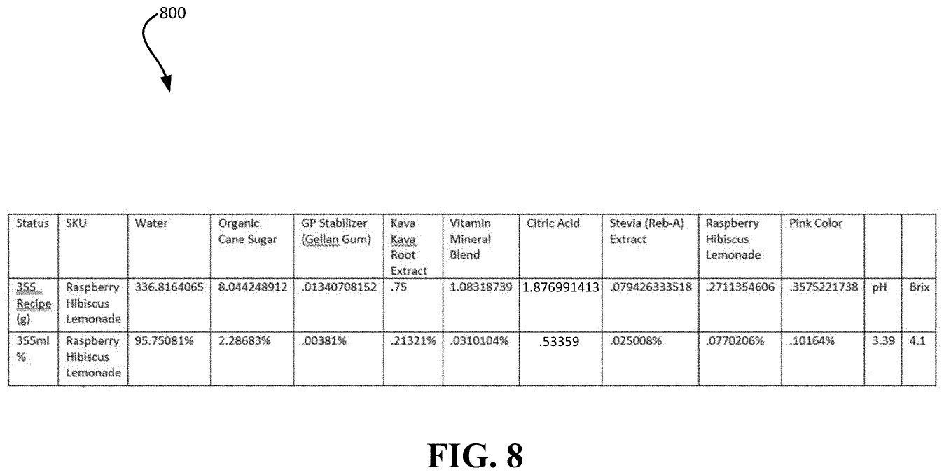

FIG. 8 is a table 800 showing a formula for a Kava-containing liquid dietary supplement having a raspberry hibiscus lemonade flavor in accordance with an embodiment of the present disclosure. Notably, the raspberry hibiscus lemonade flavored Kava dietary supplement comprises water, organic cane sugar, a stabilizer, e.g., gellan gum, Kava Kava Root extract, a vitamin and mineral blend, citric acid, and stevia. In addition, the raspberry hibiscus lemonade flavored Kava dietary supplement contains raspberry hibiscus lemonade and a pink coloring. The raspberry hibiscus lemonade flavored Kava dietary supplement has a pH of approximately 3.39 and a Brix of approximately 4.1. As noted above, Brix identifies the number of dissolved solids in the liquid via its specific gravity and is used to measure dissolved sugar, especially.

The raspberry hibiscus lemonade flavored Kava dietary supplement contains approximately 336.8164065 (approximately 337) grams of water, which makes up 95.75801% (approximately 96%) of the raspberry hibiscus lemonade flavored Kava dietary supplement. Thus, much of the raspberry hibiscus lemonade flavored Kava dietary supplement is water.

The raspberry hibiscus lemonade flavored Kava dietary supplement contains approximately 8.044248912 grams (approximately 8 grams) of organic cane sugar and makes up approximately 2.28683% (approximately 2%) of the raspberry hibiscus lemonade flavored Kava dietary supplement. As noted above, the organic cane sugar is sugar obtained directly from sugar cane

The raspberry hibiscus lemonade flavored Kava dietary supplement contains approximately 0.013400708152 grams (approximately 0.01 grams) of a stabilizer, e.g., gellan gum, which makes up approximately 0.00381% (approximately 0.004%) of the raspberry hibiscus lemonade flavored Kava dietary supplement. As noted above, gellan gum is a water-soluble anionic polysaccharide used to stabilize, thicken, add texture, and suspend other ingredients in the raspberry hibiscus lemonade flavored Kava dietary supplement.

The raspberry hibiscus lemonade flavored Kava dietary supplement contains 0.75 grams of Kava Kava root extract, which makes up approximately 0.213064% (approximately 0.2%) of the. As indicated herein, Kava Kava roots are known for their medicinal properties. For example, the Kava Kava root may be used in treating anxiety disorders, benzodiazepine withdrawal, common cold/upper respiratory tract infections, depression, epilepsy, headaches/migraines, insomnia, musculoskeletal pain, psychosis, and stress.

The raspberry hibiscus lemonade flavored Kava dietary supplement contains approximately 1.08318739 grams (approximately 1 gram) of a vitamin and mineral blend, which makes up 0.0310104% (approximately 0.03%) of the raspberry hibiscus lemonade flavored Kava dietary supplement. The vitamins and mineral blend may vary depending upon the application or use of the raspberry hibiscus lemonade flavored Kava dietary supplement.

The raspberry hibiscus lemonade flavored Kava dietary supplement contains approximately 1.876991413 grams (approximately 2 grams) of citric acid, which makes up approximately 0.53359% (approximately 0.5%) of the raspberry hibiscus lemonade flavored Kava dietary supplement. As noted above, the citric acid has a sour taste and is predominantly used as a flavoring and a preserving agent in the raspberry hibiscus lemonade flavored Kava dietary supplement.

The raspberry hibiscus lemonade flavored Kava dietary supplement contains approximately 0.079426333518 grams (approximately 0.08 grams) of Stevia extract, which makes up approximately 0.025008% (approximately 0.3%) of the raspberry hibiscus lemonade flavored Kava dietary supplement. As noted above, that Stevia is natural, unlike other sugar substitutes. It is made from a leaf related to popular garden flowers like asters and chrysanthemums.

To provide the raspberry hibiscus lemonade flavored Kava liquid dietary supplement with its raspberry hibiscus lemonade flavor, additional substances are added to the base formula. In one example of the raspberry hibiscus lemonade flavored Kava dietary supplement, approximately 0.2711354606 grams (approximately 0.3 grams), which is approximately 0.077026% (approximately 0.01%) of raspberry hibiscus lemonade is added to raspberry hibiscus lemonade flavored Kava dietary supplement. Further, pink color is added to the raspberry hibiscus lemonade flavored Kava dietary supplement. In this regard, approximately 0.3575221738 grams (approximately 0.04 grams) of pink coloring is added, which is about 0.10164 (approximately 0.1) to the raspberry hibiscus lemonade flavored Kava dietary supplement. The pH of the raspberry hibiscus lemonade flavored Kava dietary supplement is approximately 3.39 and the Brix is approximately 4.

In accordance with various embodiments, a Kava liquid dietary supplement may be made with any of a variety of sweeteners or sugar substitutes in place of or partially in place of cane sugar as disclosed above. Sweeteners or sugar substitutes may include but are not limited to stevia extract, monk fruit extract, erythritol, xylitol, allulose, other sugar alcohols, etc.

In some instances, one or more components may be referred to herein as “configured to,” “configured by,” “configurable to,” “operable/operative to,” “adapted/adaptable,” “able to,” “conformable/conformed to,” etc. Those skilled in the art will recognize that such terms (e.g., “configured to”) generally encompass active-state components and/or inactive-state components and/or standby-state components, unless context requires otherwise.

While particular aspects of the present subject matter described herein have been shown and described, it will be apparent to those skilled in the art that, based upon the teachings herein, changes and modifications may be made without departing from the subject matter described herein and its broader aspects and, therefore, the appended claims are to encompass within their scope all such changes and modifications as are within the true spirit and scope of the subject matter described herein. It will be understood by those within the art that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to claims containing only one such recitation, even when the same claim includes the introductory phrases “one or more” or “at least one” and indefinite articles such as “a” or “an” (e.g., “a” and/or “an” should typically be interpreted to mean “at least one” or “one or more”); the same holds true for the use of definite articles used to introduce claim recitations. In addition, even if a specific number of an introduced claim recitation is explicitly recited, those skilled in the art will recognize that such recitation should typically be interpreted to mean at least the recited number (e.g., the bare recitation of “two recitations,” without other modifiers, typically means at least two recitations, or two or more recitations). Furthermore, in those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). It will be further understood by those within the art that typically a disjunctive word and/or phrase presenting two or more alternative terms, whether in the description, claims, or drawings, should be understood to contemplate the possibilities of including one of the terms, either of the terms, or both terms unless context dictates otherwise. For example, the phrase “A or B” will be typically understood to include the possibilities of “A” or “B” or “A and B.”

With respect to the appended claims, those skilled in the art will appreciate that recited operations therein may generally be performed in any order. Also, although various operational flows are presented in a sequence(s), it should be understood that the various operations may be performed in other orders than those which are illustrated or may be performed concurrently. Examples of such alternate orderings may include overlapping, interleaved, interrupted, reordered, incremental, preparatory, supplemental, simultaneous, reverse, or other variant orderings, unless context dictates otherwise. Furthermore, terms like “responsive to,” “related to,” or other past-tense adjectives are generally not intended to exclude such variants, unless context dictates otherwise.

While the disclosed subject matter has been described in terms of illustrative embodiments, it will be understood by those skilled in the art that various modifications can be made thereto without departing from the scope of the claimed subject matter as set forth in the claims.

What is claimed is:

1. A liquid dietary supplement, comprising:
water;
a stabilizer; and
a water soluble Kava Kava root extract.

2. The liquid dietary of claim 1, further comprising:
a non-sugar sweetener.

3. The liquid dietary of claim 2, wherein the non-sugar sweetener includes a sugar alcohol.

4. The liquid dietary of claim 2, wherein the non-sugar sweetener includes stevia.

5. The liquid dietary of claim 2, further comprising:
a masking agent.

6. The liquid dietary of claim 2, wherein the non-sugar sweetener includes monk fruit extract.

7. The liquid dietary of claim 2, further comprising:
citric acid.

8. The liquid dietary of claim 2, further comprising:
a vitamin mineral blend.

9. The liquid dietary of claim 2, further comprising:
a flavoring blend.

10. The liquid dietary supplement of claim 9, wherein a Kava Kava root extract content is less than or equal to approximately 1.0 grams.

11. The liquid dietary supplement of claim 9, wherein a vitamin mineral blend content is approximately greater than 1 gram.

12. The liquid dietary supplement of claim 9, wherein a citric acid content is between approximately 1 gram and 3 grams.

13. The liquid dietary of claim 2, further comprising:
carbonation.

14. The liquid dietary of claim 2, further comprising:
fruit juice as a sweetener.

15. A method for preparing a liquid dietary supplement, the method comprising:
extracting kavalactones from a Kava plant;
heating water to a defined temperature;
mixing kavalactones with at least one of a sugar, a non-sugar sweetener, and a juice sweetener creating a sweetened Kava mixture;
pouring the sweetened Kava mixture, and heated water into a mixer;
mixing at high shear;
homogenizing the mixture;
pumping mixed sweetened Kava through a filter generating a slurry;
adding the slurry to water; and
agitating the slurry and the water with a mixer.

16. The method of claim 15, wherein the filter is configured to filter out particles greater than about 50 microns.

16. The method of claim 14, wherein the filter is configured to filter out particles greater than about 50 microns and the mixed sweetened Kava is pressurized through the filter.

17. The method of claim 14, further comprising:
batch pasteurization of the slurry.

18. The method of claim 14, further comprising adding Stevia to the agitated slurry.

19. The method of claim 14, further comprising adding citric acid to the agitated slurry.

20. The method of claim 14, further comprising adding gellan gum to the agitated slurry.
